(19) United States
(12) Patent Application Publication
(10) Pub. No.: US 2026/0108447 A1
(43) Pub. Date: Apr. 23, 2026

(54) RESIDENT-SKIN-FLORA-IMPROVING AGENT AND EXTERNAL-USE COMPOSITION FOR SKIN

(71) Applicant: NIHON SHOKUHIN KAKO CO., LTD., Tokyo (JP)

(72) Inventors: Moeko AIZAWA, Fuji-shi, Shizuoka (JP); Kenji YAMANE, Fuji-shi (JP)

(73) Assignee: NIHON SHOKUHIN KAKO CO., LTD., Tokyo (JP)

(21) Appl. No.: 19/117,294

(22) PCT Filed: Oct. 2, 2023
(86) PCT No.: PCT/JP2023/035896

§ 371 Date: Mar. 31, 2025

(30) Foreign Application Priority Data
Oct. 4, 2022 (JP) 2022-160070

(51) Int. Cl. A61K 8/60; A61K 31/7016; A61P 17/00; A61Q 5/02; A61Q 19/00

(52) U.S. Cl. CPC A61K 8/60; A61K 31/7016; A61P 17/00; A61Q 19/00; A61K 2800/74; A61Q 5/02; A61Q 19/007

(57) ABSTRACT

There is provided a resident-skin-flora-improving agent that is useful in keeping the skin in excellent condition. Specifically, there is provided a resident-skin-flora-improving agent in which sophorose is used. Sophorose is provided with functionality by which it is possible to promote proliferation of helpful bacteria that beneficially affect skin while suppressing growth of bacteria that could adversely affect the skin. The resident-skin-flora-improving agent can suitably be used for the object of maintaining balance of the resident skin flora and keeping the skin in excellent condition.

### TECHNICAL FIELD

The present invention relates to a resident-skin-flora-improving agent and an external-use composition for skin in which sophorose is used.

### BACKGROUND ART

A variety of symbiotic microorganisms form groups on skin, and reside therein. These microorganisms differ depending on the individual person, differ depending on the site, and vary with the season, but ordinarily a certain fixed balance is maintained. It is known that these microorganisms, which constitute the resident skin flora, affect skin function. In the skin of a healthy person, the resident skin flora maintains the pH of the epidermis at a weakly acidic level, maintains a moisturized environment, or improves resistance to pathogenic bacteria, thus keeping the skin in good condition. However, if the balance of the resident skin flora is disrupted due to, inter alia, poorness in overall state resulting from an external wound or illness, psychological stress, coldness, dryness, or unsuitable washing, then not only will the function of the skin for protecting against the external environment decline, but a variety of skin conditions could be induced and onset of disease could occur.

Examples of resident bacteria that play a role in preserving the balance of the resident skin flora and maintaining skin function include Staphylococcus epidermidis, which is known as an epidermal Staphylococcus. These resident bacteria break down sebum to produce glycerin, which serves as a moisturizing component or fatty acid that preserves the weak acidity of the skin, or produce an antibacterial peptide that suppresses proliferation of pathogenic bacteria to improve a barrier function of the skin. Additionally, Staphylococcus hominis, which is similarly known as an epidermal Staphylococcus, is also known to be a resident epidermis bacterium that promotes expression of antibacterial peptides and the like, thereby improving resistance to pathogenic bacteria.

However, resident epidermis bacteria are known to include bacteria that can become harmful with excessive proliferation. For example, Cutibacterium acnes, which is known as an acne bacillus, has been reported to be useful in protecting skin; however, these bacteria are known to excessively proliferate and serve as a factor in causing acne vulgaris (acne) to occur if the amount of sebum secreted increases or if pores become clogged.

Staphylococcus aureus, which is known as golden Staphylococcus, is known to produce a toxin serving as a factor in inflammation or breaching of the skin barrier and to be present in large quantities in persons with chapped skin, persons with dry skin, and patients with atopic dermatitis.

Corynebacterium xerosis, which is known as an actinomycete, is known to produce a substance serving as a factor in causing foul odor from sebum. Micrococcus luteus, which is similarly known as an actinomycete, is known to break down sweat and produce a malodorous substance in addition to being present in large quantities in persons with chapped skin.

As indicated above, the resident skin flora is considered to be extremely important in keeping the skin in good condition.

In relation to the resident skin flora, for example, Patent Document 1 indicates that a gluco-oligosaccharide having one or more α-1,2 glycosidic bonds presents an action and an effect for promoting proliferation of useful bacteria residing on the skin and inhibiting proliferation of undesirable bacteria, such as bacteria serving as factors in foul odor or pathogenic bacteria. Patent Document 2 discloses a resident-skin-flora-improving agent containing an isomalto-oligosaccharide and/or a sugar alcohol obtained by reducing an isomalto-oligosaccharide. Patent Document 3 discloses a resident-skin-flora-improving agent containing a xylo-oligosaccharide that contains a uronic acid residue. Patent Document 4 indicates that cellobiose or a cello-oligosaccharide-containing composition having cellobiose as a main ingredient exhibits a bacteriostatic action or a proliferation-suppressing action with respect to Staphylococcus aureus or Pseudomonas aeruginosa, but does not exhibit a bacteriostatic action or a proliferation-suppressing action with respect to Staphylococcus epidermidis. Patent Document 5 discloses a resident-skin-flora-balance-improving agent having lactosucrose as an active ingredient.

### RELATED ART DOCUMENTS

### Patent Documents

[Patent Document 1] Japanese Laid-open Patent Application No. 2002-113041
[Patent Document 2] Japanese Laid-open Patent Application No. 2005-89355
[Patent Document 3] Japanese Laid-open Patent Application No. 2007-77121
[Patent Document 4] International Publication No. 2007/037249
[Patent Document 5] International Publication No. 2021/132019

### DISCLOSURE OF THE INVENTION

### Problems the Invention is Intended to Solve

In the related art, there have been known to be trials for improving the flora of resident skin bacteria using various oligosaccharides and the like. However, there has been demand for new materials to be developed, whereby the purpose of consumer convenience can be satisfied.

It is an object of the present invention to provide a resident-skin-flora-improving agent that is useful in keeping the skin in excellent condition.

### Means for Solving the Aforementioned Problems

As a result of various investigations carried out in order to achieve the aforementioned object, the inventors perfected the present invention upon discovering sophorose as a material for selectively promoting proliferation of bacteria that beneficially affect the skin.

Specifically, according to a first aspect, the present invention provides a resident-skin-flora-improving agent having sophorose as an active ingredient.

In the present invention, the aforementioned resident-skin-flora-improving agent is preferably used for promoting proliferation of Staphylococcus epidermidis and/or Staphylococcus hominis while inducing bacteriostasis in one or more selected from the group consisting of Cutibacterium acnes, Staphylococcus aureus, Corynebacterium xerosis, and Micrococcus luteus.

Moreover, according to a second aspect, the present invention provides an external-use composition for skin, the composition containing the aforementioned resident-skin-flora-improving agent.

### Effect of the Invention

According to the present invention, using sophorose makes it possible to promote proliferation of helpful bacteria that beneficially affect skin while suppressing growth of bacteria that could adversely affect the skin.

### BRIEF DESCRIPTION OF THE DRAWINGS

FIG. 1 is a chart showing results of investigating a change in bacteria count when, in test example 1, in circumstances where various bacteria are to be cultured as resident skin bacteria under culturing conditions suited thereto, an α-gluco-oligosaccharide or a β-gluco-oligosaccharide is added to a culture medium for the bacteria and culturing is carried out;

FIG. 2 is a chart showing results of investigating a change in bacteria count when sophorose, laminaribiose, or gentiobiose prepared at high purity is added to a culture medium and culturing is carried out in culturing of Staphylococcus epidermidis as resident skin bacteria under suitable culture conditions in test example 2;

FIG. 3 is a chart showing results of investigating a change in bacteria count when sophorose prepared at high purity is added at various concentrations to a culture medium and culturing is carried out in culturing of Staphylococcus epidermidis as resident skin bacteria under suitable culture conditions in test example 3;

FIG. 4 is a chart showing results of investigating a change in bacteria count when sophorose prepared at high purity is added at various concentrations to a culture medium and culturing is carried out in culturing of various bacteria as resident skin bacteria under respectively suitable culture conditions in test example 4; and

FIG. 5 is a chart showing results of investigating a change in bacteria count when laminaribiose or gentiobiose prepared at high purity, or commercially available cellobiose, is added to a culture medium and culturing is carried out in culturing of various bacteria as resident skin bacteria under respectively suitable culture conditions in test example 5.

### MODE FOR CARRYING OUT THE INVENTION

The present invention is described in further detail below. In the present description, dashes in numeric value ranges indicate that a parameter is equal to or greater than one numeric value and equal to or less than another numeric value, and include the numeric values at both ends of the range, unless otherwise indicated. When upper-limit values and lower-limit values for numeric value ranges are indicated, the upper-limit values and lower-limit values can be combined as appropriate, and the resulting numeric value ranges are also included in the disclosure of the present description.

In the resident-skin-flora-improving agent according to the present invention, sophorose is used as an active ingredient. Sophorose (also known as 2-O-β-D-glucopyranosyl-D-glucose) is a disaccharide in which two glucose molecules are joined by a β-1,2 bond. Sophorose was initially discovered as a sugar component of a glycoside in a pigment in the pods of Styphnolobium japonicum, which is a plant in the family Fabaceae. This sugar is rarely present in the form of a free disaccharide in the natural world, and naturally is only present in very slight quantities. Sophorose is only known to be present in slight quantities in royal jelly and acid hydrolysates of starch.

The manner in which to procure the sophorose is not particularly limited. For example, the sophorose can be enzymatically prepared using a sugar transfer/condensation reaction. Specifically, as is described in detail in, e.g., Japanese Patent No. 2750374 and Japanese Patent No. 3020583, sophorose can be obtained by causing a β-glucosidase from a microorganism source to act on glucose and/or a β-gluco-oligosaccharide and using a condensation/sugar transfer reaction fulfilled by the β-glucosidase. Additionally, as is described in detail in Japanese Patent No. 7025941, sophorose can be obtained at higher yield through a method that causes 1,2-β-oligoglucan phosphorylase to act on α-glucose-1-phosphoric acid and glucose.

These methods make it possible to obtain a composition that contains sophorose. The resulting sophorose-containing composition can be subjected to treatments such as filtration, decoloration, deodorization, or desalination, as necessary. It is also permissible to implement treatments such as: extraction; centrifugation; crystallization; microorganism metabolism; fractionation carried out through chromatography using activated carbon, a porous carrier, a hydrophobic resin, a hydrophilic resin, an ion-exchange resin, an adsorption resin, or the like; dialysis; or membrane fractionation with ultrafiltration or the like. These treatments make it possible to raise the purity of the sophorose.

In the present invention, the sophorose can be used directly in a highly purified form, or can be used in the form of a sophorose-containing substance, i.e., a sophorose-containing sugar composition. In the latter case, there are no particular limitations as to the sophorose content of the sophorose-containing sugar composition in the present invention. For example, the sophorose content is preferably 10 mass % or higher, more preferably 30 mass % or higher, even more preferably 50 mass % or higher, and most preferably 80 mass % or higher, in terms of dry solid content. If the sophorose content is less than 5 mass % in terms of dry solid content, then it could not be possible for functionality of the sophorose to be sufficiently exhibited.

It is permissible to use a composition that contains sugars, oligosaccharides, or other components having different bonding patterns or degrees of polymerization as the sophorose-containing sugar composition used in the present invention. As a typical example, the sophorose-containing sugar composition can be exemplified by, inter alia, a composition that contains one or more from among sophoro-oligosaccharides (excluding sophorose), gentio-oligosaccharides, cello-oligosaccharides, laminari-oligosaccharides, and the like, which are oligosaccharides having a degree of polymerization of 2-10, in the respective amounts in terms of dry solid content indicated below.
Sophoro-oligosaccharides: 5 mass %-80 mass %
Gentio-oligosaccharides: 5 mass %-50 mass %
Cello-oligosaccharides: 5 mass %-50 mass %
Laminari-oligosaccharides: 5 mass %-50 mass %

As indicated in the examples, which shall be described later, sophorose is provided with functionality by which it is possible to promote proliferation of helpful bacteria that beneficially affect skin while suppressing growth of bacteria that could adversely affect the skin (also referred to as “bacteriostasis”). Specifically, the helpful bacteria that beneficially affect the skin are not limited. Examples thereof include: Staphylococcus epidermidis, which is known as an epidermal Staphylococcus; and Staphylococcus hominis, which is similarly known as an epidermal Staphylococcus. Examples of the bacteria that could adversely affect the skin include: Cutibacterium acnes, which is known as an acne bacillus;

Staphylococcus aureus, which is also known as golden Staphylococcus; Corynebacterium xerosis, which is known as an actinomycete; and Micrococcus luteus, which is similarly known as an actinomycete.

Because sophorose, which is provided with the functionality described above, is used as an active ingredient in the present invention, the present invention can suitably be used for the object of maintaining balance in the resident skin flora and keeping the skin in excellent condition.

Specifically, according to a given aspect, the present invention can suitably be used for the object of improving the balance of the resident skin flora. If the balance of the resident skin flora is disrupted, then the resident bacteria could excessively proliferate, or other harmful bacteria could intrude and proliferate, and a variety of skin conditions could be induced. However, the present invention can suitably be used for the object of preventing the skin from succumbing to such a state, or reducing or controlling the extent of such conditions. In this case, improving the balance of the resident bacteria can include, e.g., increasing the proportion of Staphylococcus epidermidis or reducing the proportion of Staphylococcus aureus. Staphylococcus epidermidis has an antagonistic action against pathogenic bacteria such as Staphylococcus aureus and could hinder proliferation of such harmful bacteria. Therefore, promoting proliferation of Staphylococcus epidermidis makes it possible to realize a function as a resident-skin-flora-balance-adjusting agent. Additionally, improving the balance of the resident skin flora can also include, e.g., suppressing growth of harmful bacteria such as Staphylococcus aureus without affecting the growth of helpful bacteria such as Staphylococcus epidermidis.

According to a given aspect, the present invention can suitably be used for the object of preserving the weak acidity of the skin and preventing the skin from being infected by other bacteria or drying out. For example, promoting proliferation of Staphylococcus epidermidis makes it possible for these bacteria to break down sebum to produce a glycerin serving as a moisturizing component or a fatty acid that preserves the weak acidity of the skin, or produce an antibacterial peptide that suppresses proliferation of pathogenic bacteria. Therefore, the present invention can suitably be used for the object described above.

According to a given aspect, the present invention can suitably be used for the object of suppressing generation of toxins from harmful bacteria. For example, suppressing growth of Staphylococcus aureus suppresses production of toxins by these bacteria. Therefore, the present invention can suitably be used for the object described above.

According to a given aspect, the present invention can suitably be used for the object of preventing acne, or reducing or controlling the extent of this condition. For example, suppressing growth of Cutibacterium acnes makes it possible to prevent the onset of the condition acne vulgaris (acne), in which these bacteria are a factor, or to reduce or control the extent of this condition. Therefore, the present invention can suitably be used for the object described above.

According to a given aspect, the present invention can suitably be used for the object of preventing body odor, or reducing or controlling the extent of body odor. For example, suppressing growth of Corynebacterium xerosis and Micrococcus luteus, which are known as actinomycetes, makes it possible to repress these bacteria from breaking down sebum or sweat and producing substances serving as factors in causing foul odor. Therefore, the present invention can suitably be used for the object described above.

According to a given aspect, the present invention can suitably be used for the object of preventing any tendency toward specific skin conditions, or reducing or controlling the extent of these conditions. For example, the present invention can suitably be used for the object of maintaining skin health, improving skin condition, or imparting a barrier function for preventing intrusion of pathogenic bacteria from the outside.

### (External-Use Composition for Skin)

The resident-skin-flora-improving agent provided by the present invention may be used in the form of various external-use compositions for skin, such as cosmetic products, external-use medicinal products, or quasi-medicinal products. Examples of the external-use composition for skin include cosmetic creams, milky lotions, cosmetic lotions, pack preparations, skin milks (emulsions), gel preparations, powders, lip creams, lipsticks, under-makeup, foundations, sunscreens, bath preparations, hair shampoos, hair rinses, body shampoos, body rinses, soaps, cleansing foams, ointments, patches, jelly preparations, and aerosol preparations.

In cases where the resident-skin-flora-improving agent provided by the present invention is used in the form of an external-use composition for skin, the sophorose or the sophorose-containing sugar composition described above can be prepared in respective forms using a suitable and pharmaceutically acceptable formulation carrier/ingredient in accordance with ordinary methods as appropriate. Examples of such a formulation carrier/ingredient include: glycerin, petroleum jelly, urea, hyaluronic acid, heparin, and other moisturizing agents; PABA derivatives (para-aminobenzoic acid, Escalol 507, etc.), cinnamic acid derivatives (Neo Heliopan, Parsol MCX, Sunguard B, etc.), salicylic acid derivatives (octyl salicylate, etc.), benzophenone derivatives (ASL-24, ASL-24S, etc.), dibenzoylmethane derivatives (Parsol A, Parsol DAM, etc.), heterocycle derivatives (the Tinuvins, etc.), titanium oxide, and other ultraviolet absorption/dispersion agents; edetate disodium, edetate trisodium, citric acid, sodium citrate, tartaric acid, sodium tartrate, lactic acid, malic acid, sodium polyphosphate, sodium metaphosphate, gluconic acid, and other metal blocking agents; salicylic acid, sulfur, caffeine, tannin, and other sebum-suppressing agents; benzalkonium chloride, benzethonium chloride, chlorhexidine gluconate, and other sterilization/disinfection agents; diphenhydramine hydrochloride, tranexamic acid, guaiazulene, azulene, allantoin, hinokitiol, glycyrrhizic acid and salts thereof, glycyrrhizic acid derivatives, glycyrrhetic acid, and other anti-inflammatory agents; vitamin A, the vitamin B group (B1, B2, B6, B12, and B15), folic acid, nicotinic acids, pantothenic acids, biotin, vitamin C, the vitamin D group (D2 and D3), vitamin E, ubiquinones, vitamin K (K1, K2, K3, and K4), and other vitamins; aspartic acid, glutamic acid, alanine, lysine, glycine, glutamine, serine, cysteine, cystine, tyrosine, proline, arginine, pyrrolidonecarboxylic acid, and other amino acids and derivatives thereof; retinol, tocopherol acetate, magnesium ascorbyl phosphate, ascorbic acid glucoside, arabutin, kojic acid, ellagic acid, liquid placenta extracts, and other whitening agents; butylated hydroxytoluene, butylated hydroxyanisole, propyl gallate, and other antioxidants; zinc chloride, zinc sulfate, zinc phenolate, zinc oxide, potassium aluminum sulfate, and other astrigents; glucose, fructose, maltose, sucrose, trehalose, erythritol, mannitol, xylitol, lactitol, and other sugars; and various plant extracts of licorice, chamomile, horse chestnuts, strawberry saxifrage, Chinese peony, Chinese quince, scutellaria root, phellodendron bark, coptis rhizome, Houttuynia cordata, ginkgo leaf, and the like, as well as oily ingredients, surfactants, thickeners, alcohols, powdered ingredients, pigments, and the like. These formulation carriers/ingredients may be discretionarily selected and supplied from various materials through performance of ordinarily performed processes (e.g., pulverization, milling, washing, hydrolysis, fermentation, refinement, pressing, extraction, fractionation, filtration, drying, powderization, granulation, dissolution, sterilization, pH adjustment, deodorization, decoloration, and other treatments, discretionarily selected and optionally combined), in accordance with the type and form of product to be added.

In cases where additives derived from animal-based raw materials or extracts (galenicals) from various plants are supplied to the external-use composition for skin according to the present invention, it is possible to anticipate not only protection of skin and hair but also beautifying effects such as moisturization, improvement of texture/feel, imparting of softness, relief of irritation, stress relief through aroma, cell activation (prevention of cell aging), suppression of inflammation, improvement of skin quality/hair quality, prevention and amelioration of chapped skin, new hair growth, hair restoration, prevention of hair loss, imparting of luster, cleaning, amelioration of fatigue, promotion of blood circulation, and hot-bath effects, as well as other effects such as imparting of fragrance, deodorization, thickener, antisepsis, and cushioning.

Furthermore, it is also possible to anticipate a variety of already-known beautifying and medicinal effects of the raw materials. Combining these effects makes it possible to arrive at a product from which many functional effects are anticipated in addition to the functionality for improving resident skin flora that is the object of the present invention.

In the resident-skin-flora-improving agent or the external-use composition for skin provided by the present invention, the sophorose content can be within the range from 0.01 mass % to 100 mass % in terms of dry solid content. If the sophorose content is less than 0.01 mass % in terms of dry solid content, the functionality of the sophorose may not be sufficiently exhibited. The sophorose content is more preferably 0.6 mass % or higher, and even more preferably 0.75 mass % or higher.

In cases where the resident-skin-flora-improving agent or the external-use composition for skin provided by the present invention is in the form of a liquid, a gel, a cream, or another form that contains moisture and other media, the sophorose content of such forms may be set as appropriate and is not particularly limited. As a typical example, the sophorose content may be within the range of 0.01 mg/g-400 mg/g (where the denominator represents the weight of the formulation), the range of 0.1 mg/g-100 mg/g, or the range of 1 mg/g-50 mg/g.

In cases where the resident-skin-flora-improving agent or the external-use composition for skin provided by the present invention is applied to the skin, the amount administered may be set as appropriate and is not particularly limited. For example, the amount of sophorose administered may be within the range of 0.001 mg/cm²-1 mg/cm² (where the denominator represents the surface area of the skin to which the formulation is applied), the range of 0.005 mg/cm²-0.5 mg/cm², or the range of 0.02 mg/cm²-0.05 mg/cm².

### EXAMPLES

The present invention is more specifically described via examples below, but these examples do not in any way limit the scope of the present invention.

### 1. Preparation of α-Gluco-Oligosaccharide

A glucoamylase enzyme formulation was added in an amount of 50 mg per gram of solid content to a 75 (w/w) % glucose solution. The pH of the resulting combination was adjusted to 5.0, after which the combination was held at 66° C. After the combination was reacted for 63 hours, the pH was adjusted to 4.0 and the temperature of the combination was raised to 80° C., whereby the enzyme was deactivated, and monosaccharides were removed from the resulting product through membrane fractionation to obtain an α-gluco-oligosaccharide.

### 2. Preparation of β-Gluco-Oligosaccharide

A β-glucosidase enzyme formulation was added in an amount of 9.3 mg per gram of solid content to a 60 (w/w) % glucose solution. The pH of the resulting combination was adjusted to 6.6, after which the combination was held at 60° C. After the combination was reacted for 72 hours, the pH was adjusted to 4.0 and the temperature of the combination was raised to 80° C., whereby the enzyme was deactivated, and monosaccharides were removed from the resulting product through membrane fractionation to obtain a β-gluco-oligosaccharide.

### 3. Preparation of High-Purity Sophorose

Sucrose in an amount of 1.0 M, and glucose in an amount of 1.0 M, were dissolved in a 100 mM potassium phosphate buffer solution (pH 7.0). Per milliliter of the resulting base substance, 19 mg/mL of a Bifidobacterium-scardovii-derived protein having 1,2-β-oligoglucan phosphorylase activity (i.e., the protein disclosed as “BSGP” in Japanese Patent No. 7025941) and 17 mg/mL of sucrose phosphorylase (Sigma-Aldrich) were each added in an amount of 10 μL, and the combination was reacted at 30° C. After 7 days, the combination was boiled for ten minutes to deactivate the enzymes, and the reaction liquid was condensed as appropriate and supplied to an activated carbon column to remove monosaccharides and disaccharides. Hydrochloric acid was added to the resulting liquid so as to reach a final concentration of 85 mM, and the combination was held at 80° C. for 17 hours to carry out acidolysis. The resulting decomposition sample was supplied for HPLC under the conditions indicated below, and a disaccharide fraction was recovered, to obtain high-purity sophorose.

### <HPLC Conditions>

Column: ULTRON PS-80NL (Shinwa Chemical Industries, Ltd.)
Column temperature: 50° C.
Elution liquid: Ultrapure water
Flow rate: 0.9 mL/min
Detector: Differential refractive index detector
Analysis time: 20 minutes

### 4. Preparation of High-Purity Laminaribiose

Curdlan (Mitsubishi Corp. Life Sciences Ltd.) in an amount of 8.0 g and ultrapure water in an amount of 720 mL were mixed together. 8 mL of 1 M sodium hydroxide was added to the resulting mixture, and the resulting combination was stirred for 10 minutes. 40 mL of a 1 M tris-HCl buffer solution (pH 7.0) and 8 mL of 1 M hydrochloric acid were added, and the resulting combination was stirred, after which 80 mg of a yeast-cell-wall-dissolving enzyme (“Zymolyase®,” Nacalai Tesque Inc.) was added. Cysteine was added so as to reach a final concentration of 10 mM, and the resulting combination was reacted overnight at 37° C. under vigorous stirring. The resulting material was heated at 80° C. for 10 minutes to deactivate the enzyme and then cooled to room temperature, after which centrifugation was carried out and a supernatant was recovered. The supernatant was supplied for HPLC under the same conditions as those in the preparation of the high-purity sophorose, and a disaccharide fraction was recovered, to obtain high-purity laminaribiose.

### 5. Preparation of High-Purity Gentiobiose

Using the β-gluco-oligosaccharide prepared in section “[2. Preparation of β-gluco-oligosaccharide]” above, which was mainly composed of disaccharides, as a source, fractionation was carried out using the activated carbon column to obtain high-purity gentiobiose.

### 6. Sugar Compositional Analyses

Sugar compositional analyses were carried out using HPLC. The amounts of components at each degree of polymerization were derived using the peak area of the resulting chromatographs. The gluco-oligosaccharides were analyzed in accordance with HPLC conditions A, and the high-purity samples were analyzed in accordance with HPLC conditions A and HPLC conditions B.

### <HPLC Conditions A>

Column: ULTRON PS-80NL (Shinwa Chemical Industries, Ltd.)
Column temperature: 50° C.
Elution liquid: Ultrapure water
Flow rate: 0.9 mL/min
Detector: Differential refractive index detector
Analysis time: 20 minutes

### <HPLC Conditions B>

Column: HILICpak VG-50 4E (Showa Denko KK)
Column temperature: 40° C.
Elution liquid: Acetonitrile: ultrapure water at 8:2 ratio
Flow rate: 0.6 mL/min
Detector: Charged particle detector
Analysis time: 60 minutes

Tables 1 and 2 show the results of the sugar compositional analyses.

TABLE 1
Sugar composition of gluco-oligosaccharide

| | | | | DP4 or |
|---|---|---|---|---|
| | DP1 | DP2 | DP3 | higher |
| α-gluco-oligosaccharide | 0.2 | 59.8 | 25.0 | 14.9 |
| β-gluco-oligosaccharide | 0.0 | 73.9 | 23.4 | 2.6 |

DP: Degree of polymerization
(Units : mass %)

As shown in table 1, the gluco-oligosaccharides prepared as described above both had sugar compositions mainly composed of disaccharides.

TABLE 2
Sugar composition of high-purity preparations

| | | DP2 | | | | | DP3 or |
|---|---|---|---|---|---|---|---|
| | DP1 | Gentiobiose | Sophorose | Cellobiose | Laminaribiose | Isotrehalose | higher |
| Sophorose | 0.0 | 1.2 | 93.8 | 2.4 | 0.0 | 1.6 | 1.0 |
| Laminaribiose | 0.0 | 0.0 | 0.0 | 1.3 | 96.2 | 0.0 | 1.7 |
| Gentiobiose | 0.0 | 99.8 | 0.0 | 0.0 | 0.0 | 0.0 | 0.0 |

DP: Degree of polymerization
(Units: mass %)

As shown in table 2, the high-purity preparations of sophorose, laminaribiose, and gentiobiose prepared as described above all had a purity of at least 90 mass % or higher.

### Test Example 1

An investigation was conducted in regard to a change in bacteria count when the α-gluco-oligosaccharide or the β-gluco-oligosaccharide prepared as described above was added to a culture medium and culturing was carried out in culturing of the various bacteria indicated in table 3 as resident skin bacteria under respectively suitable culture conditions.

TABLE 3
Culturing conditions for various bacteria

| Bacterial strain | Classification | Culture medium | Conditions |
|---|---|---|---|
| Staphylococcus epidermidis | Epidermal | LB culture | 37° C., standing |
| (JCM20345) | Staphylococcus | medium | |
| Staphylococcus hominis | Epidermal | SCD culture | 37° C., standing |
| (JCM31912) | Staphylococcus | medium | |
| Cutibacterium acnes | Acne bacillus | Modified GAM | 37° C., anaerobic*, |
| (JCM6425) | | culture medium | standing |
| Staphylococcus aureus | Golden | NB culture | 37° C., standing |
| (ATCC6538) | Staphylococcus | medium | |
| Corynebacterium xerosis | Actinomycete | SCD culture | 37° C., standing |
| (JCM1971) | | medium | |
| Micrococcus luteus | Actinomycete | LB culture | 28° C., shaking |
| (JCM1464) | | medium | (160 rpm) |

*Cultured under anaerobic conditions using anaerobic jar and AnaeroPack Kenki (Mitsubishi Gas Chemical Co.)

Specifically, the various bacteria were cultured overnight using respectively suitable culture media to prepare preculture solutions, and the α-gluco-oligosaccharide or the β-gluco-oligosaccharide prepared as described above was added to 3 mL of fresh culture media so as to reach a final concentration of 0.5 (w/v) %, whereupon 0.03 ml of the preculture liquids was mixed thereinto and cultured for 24 hours, and the bacterial turbidity (OD660) after culturing was measured. Additionally, glucose was added as a sugar to a culture medium and similar tests were performed to arrive at a comparative control. The results were expressed as relative values (percentage relative to non-sugar-added group), where 100% indicated the bacterial turbidity when sterilized ultrapure water was added instead of any sugar.

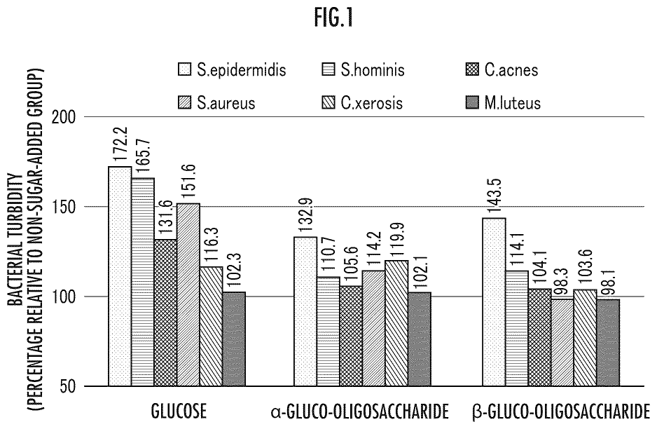

As shown in FIG. 1, the results indicated that, in the glucose-added group, proliferation of S. epidermidis and S. hominis was prominent, and proliferation of S. aureus, C. acnes, and C. xerosis was also observed, and thus selectivity for specific bacterial strains was poor. In the α-gluco-oligosccharide-added group as well, proliferation of S. aureus and C. xerosis was observed in addition to that of S. epidermidis. However, in the β-gluco-oligosccharide-added group, although proliferation of S. epidermidis and S. hominis was observed, proliferation of the other four bacterial strains was substantially not observed.

According to the above, it was clear that the β-gluco-oligosccharide mainly composed of disaccharides has an action and effect for promoting proliferation of epidermal Staphylococcus bacteria that are helpful for skin (such as S. epidermidis and S. hominis) while suppressing growth of bacteria that are harmful to the skin.

### Test Example 2

The effects of sophorose, laminaribiose, and gentiobiose, as the β-gluco-oligosccharide composed of disaccharides, for promoting growth of S. epidermidis were evaluated.

Specifically, the effect for promoting proliferation of S. epidermidis was evaluated in the same manner as in test example 1, except that sophorose, laminaribiose, or gentiobiose prepared at high purity as described above was used as the sugar to be added to the culture medium, and the amount of the sugar added to the culture medium was set to 0.3 (w/v) % or 0.5 (w/v) % in terms of the final concentration.

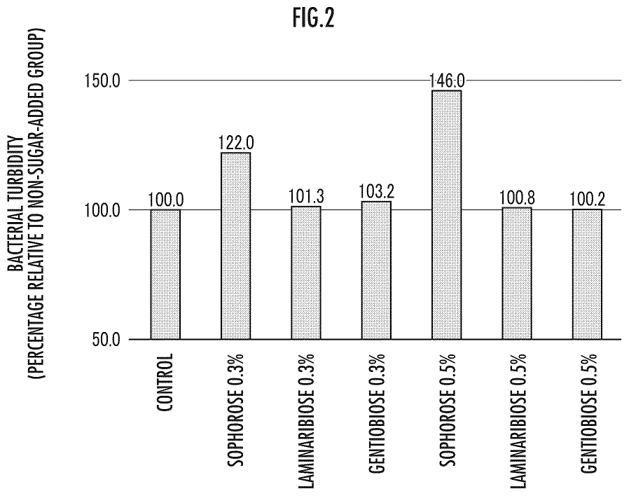

As shown in FIG. 2, the results indicated that sophorose, among the various β-gluco-oligosaccharides (disaccharides), exhibited a prominent effect for promoting proliferation of S. epidermidis. However, laminaribiose and gentiobiose did not exhibit an effect for promoting proliferation of S. epidermidis.

### Test Example 3

The effect for promoting proliferation of S. epidermidis was evaluated in the same manner as in test example 2, except that sophorose prepared at high purity as described above was used as the β-gluco-oligosaccharide composed of disaccharides, and the amount of the sophorose added to the culture medium was set to 0.1 (w/v) % or 1.0 (w/v) % in terms of the final concentration.

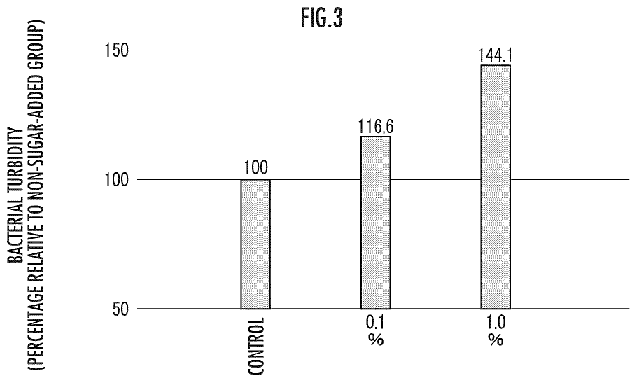

As shown in FIG. 3, the results indicated that a preparation containing sophorose at high purity was found to exhibit the effect for promoting proliferation of S. epidermidis even in the case of culturing when the preparation was added to the culture medium to yield a final concentration of 0.1 (w/v) %. However, at a final concentration of 1.0 (w/v) %, no great difference was observed from the effect for promoting proliferation of S. epidermidis that was exhibited in test example 2 when the final concentration was 0.5 (w/v) %.

### Test Example 4

The effect for promoting proliferation of S. hominis, S. aureus, C. acnes, C. xerosis, or M. luteus was evaluated in the same manner as in test example 1, except that sophorose prepared at high purity as described above was used as the β-gluco-oligosaccharide composed of disaccharides, and the amount of the sophorose added to the culture medium was set to 0.1 (w/v) %, 0.5 (w/v) %, or 1.0 (w/v) % in terms of final concentration.

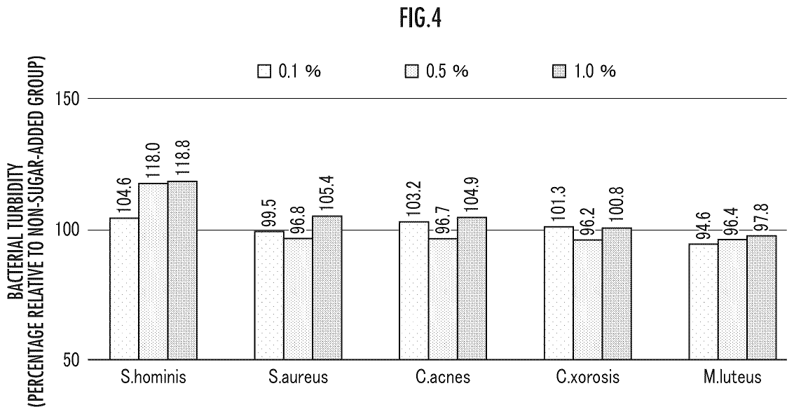

As shown in FIG. 4, the results made clear that adding a preparation containing sophorose at high purity to the culture medium so as to yield a final concentration of 0.5 (w/v) % or 1.0 (w/v) % resulted in proliferation of S. hominis increasing to 118.0% and 118.8%, respectively, as relative values (in terms of percentage relative to non-sugar-added group) with respect to a control in which no sugar was added. However, no effect for promoting proliferation of S. aureus, C. acnes, C. xerosis, or M. luteus was observed relative to the control at any of the aforementioned addition amounts.

According to the above, in consideration of all the effects of test examples 2 and 3, it was clear that sophorose, as a β-gluco-oligosaccharide composed of disaccharides, exhibited an action and effect for promoting proliferation of epidermal Staphylococcus bacteria that are helpful for skin (such as S. epidermidis and S. hominis) while repressing growth of bacteria that are harmful to the skin.

### Test Example 5

The effect for promoting proliferation of S. hominis, S. aureus, C. acnes, C. xerosis, or M. luteus was evaluated in the same manner as in test example 1, except that laminaribiose and gentiobiose prepared at high purity as described above, as well as commercially available cellobiose (Nacalai Tesque, Inc.), were used as the β-gluco-oligosaccharide composed of disaccharides, and the amount of the β-gluco-oligosaccharide added to the culture medium was set to 0.5 (w/v) % in terms of final concentration.

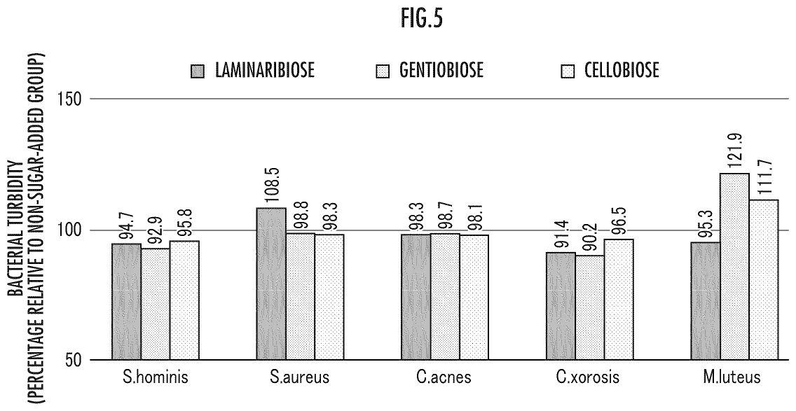

As shown in FIG. 5, the results indicated that addition of laminaribiose, gentiobiose, or cellobiose was not confirmed to yield an effect for promoting proliferation. Additionally, it was indicated that: in the laminaribiose-added group, proliferation of S. aureus increased to 108.5%, as a relative value (in terms of percentage relative to non-sugar-added group) with respect to a control in which no sugar was added; and in the gentiobiose- and cellobiose-added groups, proliferation of M. luteus during addition increased to 121.9% and 111.7%, respectively, as relative values (in terms of percentage relative to non-sugar-added group) with respect to the control in which no sugar was added.

According to the above, in consideration of all the effects of test example 2, it was clear that laminaribiose, gentiobiose, and cellobiose, as β-gluco-oligosaccharides composed of disaccharides, exhibited an inferior action and effect for promoting proliferation of epidermal Staphylococcus bacteria that are helpful for skin (such as S. epidermidis and S. hominis) while repressing growth of bacteria that are harmful to the skin, relative to the action and effect exhibited by sophorose.

### Formulary Example 1

Below is a formulary example in which sophorose is used in a cosmetic lotion as the resident-skin-flora-improving agent according to the present invention.
(Raw material name), content (mass %)
Sophorose, 0.5%
Betaine, 1.0%
Glycerin, 5.0%
1,3-butylene glycol, 10.0%
Carboxymethyldextrin sodium, 0.1%
Dipotassium glycyrrhizinate, 0.1%
Purified water, remainder

### Formulary Example 2

Below is a formulary example in which sophorose is used in a hair shampoo as the resident-skin-flora-improving agent according to the present invention.
(Raw material name), content (mass %)
Sophorose, 0.5%
Betaine, 0.5%
Sodium lauroyl aspartate solution, 45.0%
Lauroyl alanine sodium, 12.5%
Polyoxyethylene cetyl stearyl diether, 1.0%
Laureth-1,5-glycerin, 1.0%
Citric acid, 0.5%
Polyquaternium-10, 0.8%
Methylparaben, 0.2%
EDTA-2Na, 0.1%
Purified water, remainder

### Formulary Example 3

Below is a formulary example in which sophorose is used in an emulsion as the resident-skin-flora-improving agent according to the present invention.
(Raw material name), content (mass %)
Sophorose, 1.0%
Betaine, 1.0%
Glycerin, 8.0%
1,3-butylene glycol, 5.0%
Squalane, 3.0%
Olive oil, 1.0%
Cetyl octanoate, 5.0%
Stearic acid, 0.3%
Polydimethylsiloxane, 0.5%
Polysorbate 60, 0.5%
Glyceryl monostearate, 1.0%
Carboxyvinyl polymer, 0.1%
Potassium hydroxide, 0.01%
Purified water, remainder

1.-3. (canceled)

4. A method for improving resident-skin-flora, comprising applying sophorose as an active ingredient to a subject in need thereof.

5. The method for improving resident-skin-flora according to claim 4, wherein the sophorose is used for promoting proliferation of Staphylococcus epidermidis and/or Staphylococcus hominis while inducing bacteriostasis in one or more selected from the group consisting of Cutibacterium acnes, Staphylococcus aureus, Corynebacterium xerosis, and Micrococcus luteus.

6. The method for improving resident-skin-flora according to claim 4, wherein the sophorose is contained in a form of an external-use composition for skin.

7. The method for improving resident-skin-flora according to claim 5, wherein the sophorose is contained in a form of an external-use composition for skin.
